(19) United States
(12) Patent Application Publication
(10) Pub. No.: US 2012/0053549 A1
(43) Pub. Date: Mar. 1, 2012

(54) Menstural Product Collection and Disposal Device

(76) Inventor: Kim M. Knight, Conifer, CO (US)

(21) Appl. No.: 12/868,191

(22) Filed: Aug. 25, 2010

(51) Int. Cl. A61F 13/551

(52) U.S. Cl. 604/385.13

(57) ABSTRACT

A bag for use in collecting and disposing of a menstrual product includes first and second side walls having opposed side edges and end edge, respectively. Respective side and end edges are connected so that the bag defines a closed end and sides. The first side wall includes a second end edge not attached to the second side wall so as to define a bag open end opposite the bag closed end. The second side wall includes a closure portion that extends outwardly beyond the first side wall first end edge that is movable between an open configuration not covering the bag open end and a closed configuration covering the open bag end. The first and second side walls are constructed of a flexible material such that inside surfaces of the first and second side walls are movable through the open end to become exterior surfaces.

### BACKGROUND OF THE INVENTION

This invention relates generally to containment products for disposing of blood materials or other articles contaminated with blood and, more particularly, to a menstrual product collection and disposal device.

Removing a tampon or sanitary napkin is a necessary task for a woman during her monthly menstrual cycle that may be messy, uncomfortable, and poses exposure issues such as contamination. More particularly, contamination may include transfer of germs from a blood soaked menstrual article, such as a tampon or pad, onto a woman's hands or onto those of another person if exposure is in a public restroom. Further, contamination may occur from disposal of a menstrual product in a public trash receptacle.

Various products have been proposed in the art for containing a feminine product after use for disposal. Although assumably effective for their intended purposes, the existing products and prior patent proposals do not provide a structure which a woman can use to remove a menstrual product such as a pad or tampon and secure it into a containment device without risk of contamination or exposure to blood.

Therefore, it would be desirable to have a device for collecting and disposing of a menstruation product without risk of blood contamination or exposure. Further, it would be desirable to have a collection and disposal device in the form of a bag that may first be used to grasp a menstruation product and then turned “inside-out” and sealed for disposal.

### SUMMARY OF THE INVENTION

A bag for use in collecting and disposing of a menstrual product includes first and second side walls having opposed side edges and an end edge, respectively. Respective side and end edges are connected so that the bag defines a closed end and sides. The first side wall includes a second end edge not attached to the second side wall so as to define a bag open end opposite the bag closed end. The second side wall includes a closure portion that extends outwardly beyond the first side wall first end edge that is movable between an open configuration not covering the bag open end and a closed configuration covering the open bag end. The first and second side walls are constructed of a flexible material such that inside surfaces of the first and second side walls are movable through the open end to become exterior surfaces.

Therefore, a general object of this invention is to provide a menstruation product collection and disposal device that eliminates the risk of exposure to contaminants associated with blood.

Another object of this invention is to provide a menstruation product collection and disposal device, as aforesaid, that is easy and convenient to use.

Still another object of this invention is to provide a menstruation product collection and disposal device, as aforesaid, that is attractive, quiet in use, and includes anti-bacterial properties.

Yet another object of this invention is to provide a menstruation product collection and disposal device, as aforesaid, that is economical to manufacture.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a menstrual product collection and disposal device according to a preferred embodiment of the present invention;

FIG. 2 is a perspective view of the collection and disposal device as in FIG. 1 shown in a disposal configuration; and

FIG. 3 is a perspective view of the collection and disposal device as in FIG. 1 in an inverted collection configuration.

### DESCRIPTION OF THE PREFERRED EMBODIMENT

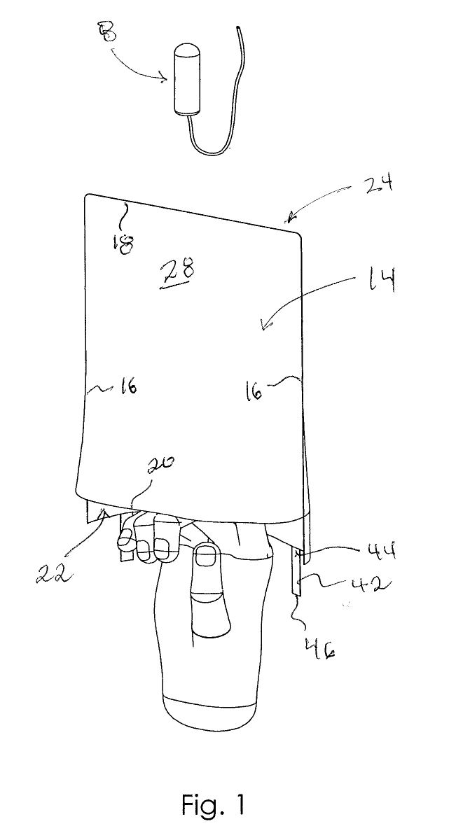

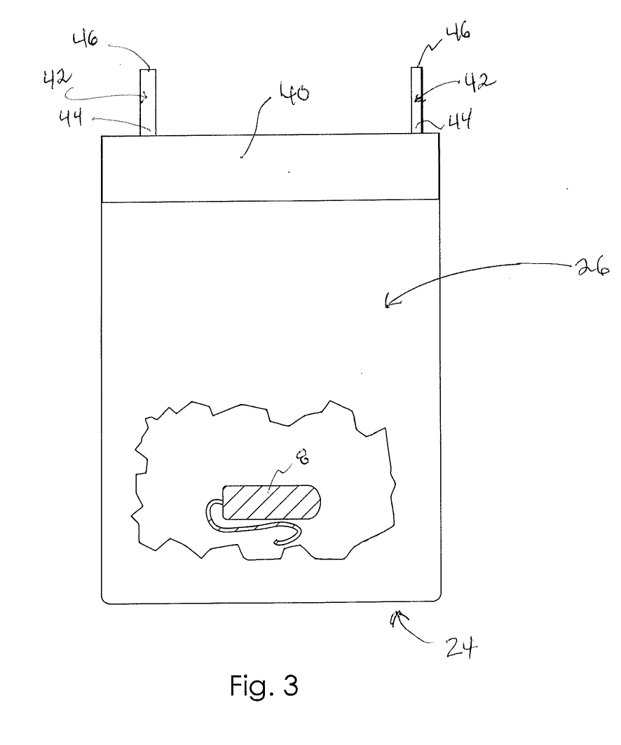

A menstrual product collection and disposal device according to a preferred embodiment of the present invention will now be described with reference to FIGS. 1 to 3 of the accompanying drawings. The collection and disposal device 10 includes a bag 12 having opposed first and second side walls 14 defining a generally open end 22 and an opposed closed end 24. The second side wall includes a construction that is substantially similar to that of the first side wall except as specifically noted below; therefore, like parts of the side walls 14 will be referenced using the same reference numerals.

More particularly, the first side wall 14 includes opposed side edges 16 and a first wall end edge 18. The second side wall includes like elements. Respective side edges 16 and end edges 18 of the side walls 14 are fixedly connected such as by having an integral construction, by heat sealing, or by other suitable means of attachment. Accordingly, the bag 12 defines closed sides and a closed end 24. The first side wall 14 includes a second end edge 20 that is not connected to the second side wall so that the bag 12 defines the open end 22 opposite the bag closed end 24.

In addition, the second side wall includes a closure portion 40 that extends outwardly beyond the first end edge 18 of the first side wall 14. The closure portion 40 is constructed of a flexible material that is movable between an open configuration not covering the bag open end 22 and a closed configuration covering the bag open end 22. At least one adhesive strip 42 is attached to a free edge of the closure portion 40. Preferably and as shown in the drawings, a pair of spaced apart adhesive strips 42 are included, each strip 42 having a proximal end 44 fixedly connected to the closure portion 40 and a distal end 46 extending away from the closure portion 40 free edge.

All portions of the bag 12 may be constructed of a liquid impervious or non-porous material such as thin plastic or other suitable material. However, it is possible that only selected surfaces of the side walls 14 be non-porous and liquid impervious. Specifically, the outer surfaces 28 of the side walls 14 may be constructed of or coated with a non-porous material. Such a construction would be adequate in that it is with the outer surfaces 28 that a menstruation product, such as a tampon 8 or pad, would be grasped. Then, the outer surfaces 28 may be inverted to the interior of the bag 12 for permanently containing the menstruation product, as will be further discussed below.

In addition, some or all of the side walls 14 and closure portion 40 may be constructed of a biodegradable material. Preferably, the side walls 14 of the bag 12 have a thin construction so as provide sufficient tactile feedback to a user who needs to feel a menstruation product therethrough in use, as will be described further below. In another aspect of the invention, the sidewalls 14 of the bag 12 include an anti-bacterial or antiseptic material. More particularly, the walls may be impregnated with an anti-bacterial material during manufacturing or be coated therewith. Accordingly, the anti-bacterial material or coating further minimizes the risk of contamination from the presence of blood.

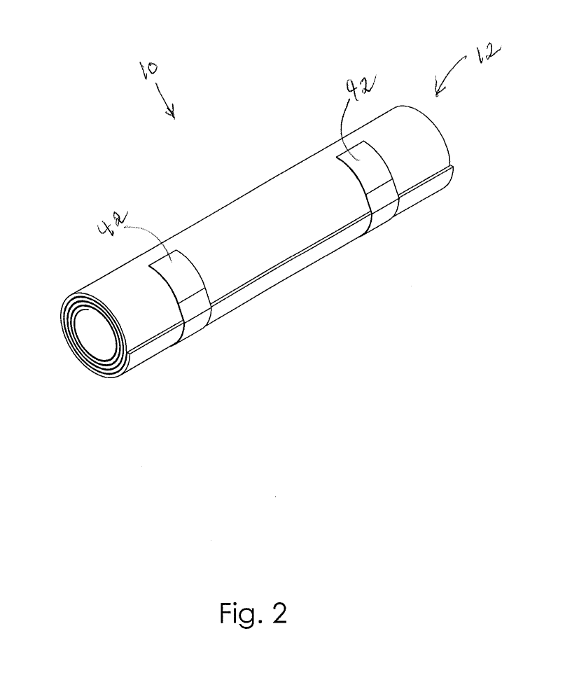

In use, the present invention may be utilized to remove a menstruation product 8, such as a tampon or pad, from a woman's body or underwear, respectively. Specifically, the device 10 may be packaged first in a rolled up configuration as shown in FIG. 2 and then unrolled when a menstruation product 8 needs to be contained and disposed of. A user may then insert her hand through the open end 22 of the bag 12 as shown in FIG. 2. The thin construction of the side walls 14 enables a user to feel a tampon 8 or pad and, therefore, to grasp it with the respective outer surfaces 28 of side walls. While grasping the inner surfaces 26 of the side walls and the feminine product 8, the user may then pull the closed end and inner surfaces through the open end 22—thus inverting the entire bag 12. It should be apparent that this action will make the inner surfaces become outer surfaces and vice versa. Further, this inversion causes the menstruation product 8 to be contained without the user ever having to physically touch it with her bare hands. Finally, the closure portion 40 may be moved to close the open end 22 of the bag 12, the bag 12 may be rolled up (FIG. 2), and the adhesive strips 40 may be adhered so as to fully enclose the bag 12 for disposal in a trash receptacle.

It is understood that while certain forms of this invention have been illustrated and described, it is not limited thereto except insofar as such limitations are included in the following claims and allowable functional equivalents thereof.

What is claimed is:

1. A bag for use in collecting and disposing of a menstrual article, comprising:
a first side wall having opposed side edges and a first end edge;
a second side wall having opposed side edges and a first end edge, wherein respective side edges and respective first end edges of said first and second side walls are sealingly connected such that said bag defines a closed end and closed sides;
wherein said first side wall includes a second end edge that is not attached to said second side wall such that said bag defines a bag open end opposite said bag closed end;
wherein said second side wall includes a closure portion that extends outwardly beyond said first side wall first end edge that is movable between an open configuration not covering said bag open end and a closed configuration covering said open bag end;
wherein said first and second side walls are constructed of a flexible material such that inner surfaces of said first and second side walls are movable through said open end to become outer surfaces.

2. The bag as in claim 1, further comprising at least one adhesive strip having a proximal end fixedly attached to a free end of said closure portion and a distal end extending away therefrom.

3. The bag as in claim 2, wherein said first and second side walls are constructed of a material that is non-porous.

4. The bag as in claim 1, wherein said outer surface includes a non-porous material.

5. The bag as in claim 3, wherein said first and second side walls are constructed of a biodegradable material.

6. The bag as in claim 1, wherein said first and second side walls are constructed of a biodegradable material.

7. The bag as in claim 5, wherein said first and second side walls include a thin construction that enables tactile feedback to a user grasping an article with said first and second side walls.

8. The bag as in claim 1, wherein said first and second side walls include a thin construction that enables tactile feedback to a user grasping an article with said first and second side walls.

9. The bag as in claim 5, wherein said first and second side walls include an anti-bacterial material.

10. A bag for use in collecting and disposing of a menstrual article, comprising:
a first side wall having opposed side edges and a first end edge;
a second side wall having opposed side edges and a first end edge, wherein respective side edges and respective first end edges of said first and second side walls are sealingly connected such that said bag defines a closed end and closed sides;
wherein:
said first and second side walls include inner and outer surfaces, respectively, at least said respective outer surfaces being non-porous;
said first side wall includes a second end edge that is not attached to said second side wall such that said bag defines a bag open end opposite said bag closed end;
said second side wall includes a closure portion that extends outwardly beyond said first side wall first end edge that is movable between an open configuration not covering said bag open end and a closed configuration covering said open bag end;
said first and second side walls are constructed of a flexible material such that said respective inner surfaces of said first and second side walls are selectively movable through said open end so as to be inverted with said respective outer surfaces; and
at least one adhesive strip having a proximal end fixedly attached to a free end of said closure portion and a distal end extending away therefrom.

11. The bag as in claim 10, wherein said first and second side walls are constructed of a biodegradable material.

12. The bag as in claim 11, wherein said first and second side walls include a thin construction that enables tactile feedback to a user grasping an article with said first and second side walls.
